(19) United States
(12) Patent Application Publication
(10) Pub. No.: US 2025/0381293 A1
(43) Pub. Date: Dec. 18, 2025

(54) PEPTIDES AND MAGNETIC PARTICLE-PEPTIDE CONJUGATES FOR PREVENTION OR TREATMENT OF GLAUCOMA

(71) Applicant: THE GOVERNORS OF THE UNIVERSITY OF ALBERTA, Edmonton (CA)

(72) Inventors: Larry UNSWORTH, Edmonton (CA); Mehdi GHAFFARI SHARAF, Edmonton (CA); Karim DAMJI, Clifton (PK)

(21) Appl. No.: 19/130,228

(22) PCT Filed: Nov. 15, 2022
(86) PCT No.: PCT/CA2022/051685

§ 371 Date: May 15, 2025

(51) Int. Cl. A61K 47/69; A61K 41/00; A61K 47/64; A61P 27/02; C07K 7/08

(52) U.S. Cl. CPC A61K 47/6923; A61K 41/00; A61K 47/64; A61P 27/02; C07K 7/08

(57) ABSTRACT

Disclosed are peptides having specific affinity to exfoliation syndrome (XFS) deposits in a mammalian eye, and magnetic particle-peptide conjugates. Also disclosed are methods and uses of such magnetic particle-peptide conjugates to displace and remove XFS deposits, to prevent or treat XFS or exfoliation glaucoma (XFG).

### FIELD OF THE INVENTION

The present invention relates to peptides which bind specifically to exfoliation syndrome material deposits, magnetic particle-peptide conjugates, and their use in the prevention and/or treatment of exfoliation syndrome glaucoma.

### BACKGROUND

Exfoliation syndrome (XFS), also known as pseudoexfoliation, is a common identifiable cause of glaucoma. XFS is commonly considered an age-related disease that significantly affects the homeostasis of the human eye through the formation of small deposits of white materials throughout the anterior segment of an eye. Although their precise composition is unknown, these XFS deposits are considered to be amyloid-like fibrils, with varied thicknesses, embedded in a fibrillogranular matrix of glycoprotein-proteoglycan crosslinks. When found in the trabecular meshwork, these fibrillar deposits are thought to impede the outflow of aqueous humor and cause large fluctuations in intraocular pressure (IOP) that ultimately leads to irreversible blindness, namely, exfoliation glaucoma (XFG). Despite best clinical practice, IOP levels for patients with XFG are unpredictable and hard to control. XFS has been also been causally related to lens subluxation, zonular instability, blood-aqueous barrier impairment, and several intraoperative and postoperative complications that occur during ocular treatments. Moreover, there is a body of evidence suggesting that XFS is a systemic disease, which presents in blood vessels, lungs, skin, gallbladder, heart, meninges, and it is a potential risk factor for other clinical complications such as coronary artery disease, cerebrovascular disease, and renal artery stenosis.

XFS deposits are not removed through normal regulatory processes necessary for ocular homeostasis and curative pharmacotherapy to prevent, break down, or remove these materials has not yet been achieved.

### SUMMARY OF THE INVENTION

In one aspect, disclosed are novel peptides that may differentiate between exfoliative and non-affected regions of the human lens capsule, and specifically bind to XFS deposits. In some embodiments, the peptides comprise or consist essentially of an amino acid sequence selected from the group consisting of LPSYNLHPHVPP [SEQ ID NO. 1], IPLLNPGSMQLS [SEQ ID NO. 2], and variants or modified derivatives thereof.

In some embodiments, such peptides may be conjugated to magnetic particles (MPs) to target and remove exfoliation deposits from the anterior human lens capsule. The MP-peptide conjugates described herein have specific affinity to XFS materials and provide a therapeutic approach for XFS whereby removal of deposits of XFS aggregates from the anterior chamber of affected eyes may help prevent or manage exfoliation related glaucoma. The MP-peptide conjugates may generate enough mechanical force to remove exfoliation deposits from the lens capsule when exposed to a magnetic field, such as a low-frequency rotating magnetic field (e.g. 5000 G, 20 Hz).

In another aspect, disclosed are methods for the treatment of exfoliation glaucoma comprising targeting of exfoliation deposits with a MP-peptide conjugate, and removal of the exfoliation deposits by application of a magnetic field. In some embodiments, the method may be applied to all tissues within the anterior segment of the eye.

In another aspect, disclosed are MP-peptide conjugates for use in a method for the treatment of exfoliation glaucoma, wherein the MP-peptide conjugates target exfoliation deposits, and may be displaced and/or removed by application of a magnetic field.

### BRIEF DESCRIPTION OF THE DRAWINGS

In the drawings, each of the embodiments depicted are but one of a number of possible arrangements utilizing the fundamental concepts of the present invention.

FIG. 1. Localization of labeled phage-displayed peptides on the human lens capsule having XFS materials. M13 phages with XFS material-targeting peptides displayed on coat protein pIII and wild-type M13 phages were labeled with Cy5 fluorescent dye and incubated with human lens capsule containing XFS materials. Phages with displayed p-LPS (A) and p-IPL (C) peptides on their surface and labeled with Cy5 dye were selectively bound to the XFS materials on the lens capsule. Wild-type phages labeled with Cy5 dye (control) did not show any specific interaction with XFS materials (E). The presence of XFS materials on the lens capsule was confirmed in the bright field mode of the microscope (B, D, and F). Arrows show the exfoliative zones on the human lens capsule. (Scale bars=100 μm).

FIG. 2. Conjugation of peptides to MPs. (A) Schematic illustration of MPs with and without peptide conjugates. (B) Surface zeta potential measurements and (C) FTIR spectra of MPs before and after peptide conjugation to MPs. a) Azide-functionalized MPs without peptide conjugates, b) MP-p-IPL, c) MP-p-LPS. (* represents p<0.05, data represent mean±1 SD, n≥3).

FIG. 3. Competitive labeling of MPs with TAMRA dye. Peptide-conjugated MPs and azide-functionalized MPs without conjugated peptides were labeled with TAMRA dye. (A, D) Control particle clumps having free azide groups showed noticeably higher fluorescence under the microscope. MPs conjugated to p-IPL (B, E) and p-LPS (C, F) showed no fluorescence under the microscope. Top images were taken in the fluorescence mode using TRITC filter and bottom images were taken in the bright field mode to confirm the presence of particles and clumps of particles. (Scale bars=50 μm).

FIG. 4. Targeting of XFS materials on the surface of the human lens capsule with MP-peptide conjugates. (A) Illustration of the anterior lens capsule showing general pattern of XFS deposits on its surface. (B) MP-p-IPL showed specific binding to XFS deposits on both central and the peripheral zones of tissue having XFS materials. The non-XFS area of lens capsule (blank spots) showed less or no particles compared to the exfoliated area. (C) MP-p-LPS also showed specific targeting of XFS materials on the surface of the lens capsule. (D) Scrambled MP-p-IPL interacted non-specifically with both XFS deposits and the area of lens capsule surface without XFS deposits. (E) Scrambled MP-p-LPS also showed non-specific interaction with XFS materials. (F) MP without peptide conjugates. As shown in the image, there is a high amount of XFS materials on the central part of the human lens capsule, however, there is not enough binding of MPs to the XFS area. Arrows indicate approximate regions of XFS materials in each tissue sample.

FIG. 5. Effect of magnetic pins on the magnetized XFS materials. Human lens capsules having XFS materials were incubated with MP-peptide conjugates and subsequently, magnetic behavior of XFS aggregates was studied using magnetic and non-magnetic tools. (A, B) Large aggregates of XFS materials covered with MP-p-IPL were pulled by magnetic pin in different directions. (C, D) XFS materials in the center of lens capsule covered with MP-p-LPS were attracted to the magnetic pin. However, in the absence of a magnetic field (i.e. non-magnetic needle) no attraction was observed. (E, F) Control studies with non-magnetic needles showed that XFS materials covered with MP-p-IPL did not react to the non-magnetic tool. (G) XFS materials (as indicated by the arrows) in the center of the human lens capsule covered with MP-peptide conjugates before application of magnetic pin. (H) Edges of the same XFS deposits in the central zone of the lens capsule being pulled towards the applied magnetic field through a magnetized pin.

FIG. 6. Effect of rotating magnetic field on XFS materials. (A-C) Control XFS lens capsules treated with rotating magnetic field without MPs. (D-F) XFS lens capsules interacted with MP-p-IPL. (G, H) XFS lens capsules interacted with MP-p-LPS. In each raw, images numbered as “I” represent tissues before treated with rotating magnetic field, images numbered as “II” represent XFS lens capsule after 3 hr treatment with rotating magnetic field, and images numbered as “III” represent lens capsules after buffer irrigation over the tissue.

FIG. 7. Mean intensity measurements of XFS lens capsules incubated with MP-peptide conjugates. (Control 1-Control 3) Control tissues incubated with BSS and treated with rotating magnetic field. (MP-p-IPL) Representative mean intensity values measured using images of XFS lens capsules interacted with MP-p-IPL. (MP-p-LPS) Mean intensity values of different lens capsules interacted with MP-p-LPS. Both groups of graphs related to test samples showed that intensities were decreased due to the removal of XFS materials from the surface of lens capsules. In each graph, column “I” represents intensity value of the images of the surface of lens capsule before treating with rotating magnetic field, column “II” represents values after 3 hr treatment with the rotating magnet, and column “III” represents intensities after applying BSS irrigation over the control XFS lens capsules. In all images, intensity values have been reported after background subtraction.

FIG. 8. MTT colorimetric assay of viable hTM cells in the presence of XFS-specific peptides in free and conjugated form. (Left) MTT assay conducted using peptide-conjugated MPs. (Right) MTT assay results after incubation of hTM cells for 24 hr with 0.5 mM and 1 mM free peptide solutions. The optical density was measured at 570 nm after 24 hr incubation of hTM cells with each test sample. (Data represent mean±1 SD, n≥3, no statistical difference was observed between the means).

FIG. 9. Electrophoretic analyses of the apoptotic chromosomal DNA fragmentation of hTM cells. (A) Isolated DNA from hTM cells after being incubated with MP-peptide conjugates for 24 hr. lane 1: 1 Kb plus DNA ladder, lane 2: Control, hTM cells treated with water, lane 3: Extracted DNA of hTM cells incubated with 100 μg MP-p-IPL for 24 hr, lane 4: Extracted DNA of hTM cells incubated with 100 μg MP-p-LPS for 24 hr, lane 5: 100 bp plus DNA ladder. (B) Isolated DNA from hTM cells incubated with free XFS-targeting peptide solutions. Lane 1: 1 Kb plus DNA ladder, lane 2: 100 bp plus DNA ladder, lane 3: Control, hTM cells treated with water, lane 4: hTM cells incubated with 1 mM MP-p-IPL for 24 hr, lane 4: hTM cells incubated with 1 mM MP-p-LPS for 24 hr.

### DETAILED DESCRIPTION OF EMBODIMENTS

Unless defined otherwise, all technical and scientific terms used herein have the same meaning as commonly understood by those of ordinary skill in the art, such as in the arts of peptide chemistry, cell culture and phage display, nucleic acid chemistry and biochemistry. Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, NY; Ausubel et al., Short Protocols in Molecular Biology (1999) 4th ed., John Wiley & Sons, Inc.), which are incorporated herein by reference.

As used herein, “XFS deposit” means the same as “XFS materials” and refers to deposits of white materials throughout the anterior segment of an eye, which deposits are associated with XFS or XFG.

As used herein, “peptide” means at least 5 amino acids. In one aspect, a peptide is 5-35 amino acids, for example, 5, 6, 7, 8, 9, 10, 15, 20, 25, 30 or 35 amino acids. In another embodiment, a peptide is 8-30 amino acids (for example, 8, 9, 10, 11, 12, 13, 14, 15, 16, 17, 18, 19, 20, 25 or 30 amino acids). In another embodiment, a peptide is 10-15 amino acids, such as 12 amino acids. A peptide can include D-amino acids, non-peptide or pseudo-peptide linkages and peptidyl mimics. In addition, the peptide and peptide mimics can be modified, e.g. glycosylated or methylated. Synthetic mimics of targeting peptides are also included.

The term “magnetic particle” means a particle which obtains a magnetic moment when placed in a magnetic field. Thus, it can be displaced.

The following abbreviations are used herein: 1) XFS: exfoliation syndrome, 2) hTM cells: human trabecular meshwork cells, 3) hAH: human aqueous humor, 4) IOP: intraocular pressure, and 5) MP: magnetic particle.

### Peptides

Disclosed are peptides having a selective affinity to XFS materials in a mammalian eye, such as a human eye. Preferred examples of these peptides were discovered by an ex vivo panning procedure to explore targeting peptides for the XFS materials using a phage display technique. The selective affinity of phage-displayed peptides was confirmed through ex vivo studies using human lens capsule and fluorescently labeled phages.

In some embodiments, the peptides comprise or consist essentially of an amino acid sequence selected from the group consisting of LPSYNLHPHVPP [SEQ ID NO. 1], IPLLNPGSMQLS [SEQ ID NO. 2], and variants thereof which comprise an amino acid substitution which substantially maintains selective affinity to XFS materials. Acceptable amino acid substitution at any given position may be determined following the results of an alanine scan and selection outputs which permits well tolerated substitutions at the selected position. Selective affinity may be observed by staining human lens capsules with XFS materials with fluorescently labeled phages displaying the variant peptides. Variants which substantially maintain selective affinity to XFS materials may retain at least 50, 60, 70, 80 or 90% binding affinity to XFS materials, using a comparative assay such as those described herein.

It will be appreciated that modified derivatives of the peptides as defined herein are within the scope of the present invention. Examples of such suitable modified derivatives include one or more modifications selected from: N-terminal and/or C-terminal modifications; replacement of one or more amino acid residues with one or more non-natural amino acid residues (such as replacement of one or more polar amino acid residues with one or more isosteric or isoelectronic amino acids; replacement of one or more non-polar amino acid residues with other non-natural isosteric or isoelectronic amino acids); addition of a spacer group; replacement of one or more oxidation sensitive amino acid residues with one or more oxidation resistant amino acid residues; replacement of one or more amino acid residues with an alanine, replacement of one or more L-amino acid residues with one or more D-amino acid residues; N-alkylation of one or more amide bonds within the bicyclic peptide ligand; replacement of one or more peptide bonds with a surrogate bond; peptide backbone length modification; substitution of the hydrogen on the alpha-carbon of one or more amino acid residues with another chemical group, modification of amino acids such as cysteine, lysine, glutamate/aspartate and tyrosine with suitable amine, thiol, carboxylic acid and phenol-reactive reagents so as to functionalize said amino acids, and introduction or replacement of amino acids that introduce orthogonal reactivities that are suitable for functionalization, for example azide or alkyn-group bearing amino acids that allow functionalization with alkyn or azide-bearing moieties, respectively.

In one embodiment, the modified derivative comprises replacement of one or more amino acid residues with one or more non-natural amino acid residues. In this embodiment, non-natural amino acids may be selected having isosteric/isoelectronic side chains which are neither recognized by degradative proteases nor have any substantial adverse effect upon target affinity.

Alternatively, non-natural amino acids may be used having constrained amino acid side chains, such that proteolytic hydrolysis of the nearby peptide bond is conformationally and sterically impeded. In particular, these concern proline analogues, bulky sidechains, C-disubstituted derivatives (for example, aminoisobutyric acid, Aib), and cyclo amino acids, a simple derivative being amino-cyclopropylcarboxylic acid.

It will be appreciated that salt forms are within the scope of this invention, and references to peptides include the salt forms of said peptides. Such salts can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two. Acid addition salts (mono- or di-salts) may be formed with a wide variety of acids, both inorganic and organic, which are well known in the art. One particular group of salts consists of salts formed from acetic, hydrochloric, hydroiodic, phosphoric, nitric, sulfuric, citric, lactic, succinic, maleic, malic, isethionic, fumaric, benzenesulfonic, toluenesulfonic, sulfuric, methanesulfonic (mesylate), ethanesulfonic, naphthalenesulfonic, valeric, propanoic, butanoic, malonic, glucuronic and lactobionic acids. One particular salt is the hydrochloride salt. Another particular salt is the acetate salt.

If the peptide is anionic, or has a functional group which may be anionic (e.g., —COOH may be —COO″), then a salt may be formed with an organic or inorganic base, generating a suitable cation. Examples of suitable inorganic cations include, but are not limited to, alkali metal ions such as Li+, Na+ and K+, alkaline earth metal cations such as Ca2+ and Mg2+, and other cations such as Al3+ or Zn+. Examples of suitable organic cations include, but are not limited to, ammonium ion (i.e., N¾+) and substituted ammonium ions (e.g., NH3R+, NH2R2, NHR3+, NR4).

The peptides disclosed herein can be made using conventional solid-phase synthesis from amino acid starting materials, which may include appropriate protecting groups as is known in the art. These methods for making peptides are well known in the art.

### Magnetic Particle Conjugates

The XFS-targeting peptides disclosed herein may be conjugated to magnetic particles. The conjugates bind to XFS materials and liberate them under an induced magnetic field.

In some embodiments, the MPs may comprise iron oxide particles (Fe3O4) or similar magnetic material such as maghemite γFe2O3 or ferrites. Such magnetic particles may be conveniently functionalized and are less sensitive to oxidation than pure metals. Furthermore, iron oxide-based MPs have higher biocompatibility than other magnetic materials including nickel and cobalt, which makes them more favorable candidates for biomedical applications. The magnetic particles may be of any size or shape which is conducive to introduction to the eye. For example, spherical particles having a diameter of about 1 μm may be suitable. In some embodiments, the particles may comprise nanoparticles, having diameters less than about 1 um, 500 nm, or 100 nm.

In some embodiments, XFS-targeting peptides may be conjugated to MPs, including iron oxide MPs, using any suitable chemistry, such as azide-alkyne cycloaddition click chemistry. Various techniques for conjugating peptides to magnetic particles or nanoparticles are well known to those skilled in the art. FTIR analysis and zeta potential measurements are used to confirm the conjugation of peptides to MPs, and competitive labeling of MPs using alkyne-modified fluorophore may also confirm the attachment of peptides to the particles.

### Therapeutic Use

Disclosed are methods for the treatment of exfoliation glaucoma comprising targeting of exfoliation materials with MP-peptide conjugates, and removal of the exfoliation materials by application of a magnetic field, a magnetic field, such as a low-frequency rotating magnetic field (e.g. 5000 G, 20 Hz). In some embodiments, the method may be applied to all tissues within the anterior segment of the eye.

The strength of the magnetic field may be chosen to effectively displace the MP-peptide conjugates and remove the XFS deposits from the lens capsule surface. In some embodiments, the magnetic field may be between about 1000 G to about 10,000 G, such as 5000 G.

In some embodiments, following application of the magnetic field, the anterior segment of the eye may be flushed or irrigated, which may enhance the removal of XFS deposits. Experimental results described below demonstrate that irrigation after MP treatment leads to enhanced removal of XFS materials from the surface of ex vivo lens capsules. Thus, upon displacement of XFS materials with a magnetic field, commonly used irrigation/aspiration systems can remove large and small XFS materials from the anterior chamber of the eye. Therefore, this technique may substantially eliminate XFS materials from the majority of surfaces in the anterior ocular chamber. Displacement with a magnetic field and flushing or irrigation may be alternated and repeated as necessary or desired.

Compared to the other targeting approaches, peptide-based therapeutic strategies benefit from a lower immunogenicity profile, higher binding affinity, and increased specificity related to the small peptide molecules relative to other drug compounds. XFS-targeting MP-peptide conjugates disclosed herein show selective and high affinity to XFS materials on the human lens capsule. Although XFS materials have been clinically characterized with a general deposition pattern on the lens capsule, there are individual variations from patient to patient. Despite those variations, the peptides disclosed herein had acceptable selective binding to XFS materials in most of the lens capsule areas associated with XFS materials. Peptide modified MPs may remove XFS materials from a wide range of patient samples when an external magnetic field was applied. Accordingly, the MP-peptide conjugates described herein may provide a minimally invasive therapeutic strategy for treating XFS that may affect the onset and/or the course of glaucoma.

Biocompatibility of targeting peptides with and without conjugated magnetic particles was confirmed using MTT cell toxicity assay, live/dead cell viability assay, and DNA fragmentation studies on primary cultured human trabecular meshwork cells.

Therapeutic and prophylactic uses of the peptides and/or MP-peptide conjugates disclosed herein involve the administration of such peptides or MP-peptide conjugates to a recipient mammal, such as a human. Substantially pure peptides of at least 90 to 95% homogeneity are preferred for administration to a mammal, and 98 to 99% or more homogeneity is most preferred for pharmaceutical uses, especially when the mammal is a human. Once purified, partially or to homogeneity as desired, the selected peptides may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

Generally, the present peptides may be utilized in purified form together with pharmacologically appropriate carriers. Typically, these carriers include aqueous or alcoholic/aqueous solutions, emulsions or suspensions, any including saline and/or buffered media. Parenteral vehicles include sodium chloride solution, Ringer's dextrose, dextrose and sodium chloride and lactated Ringer's. Suitable physiologically-acceptable adjuvants, if necessary to keep a peptide complex in suspension, may be chosen from thickeners such as carboxymethylcellulose, polyvinylpyrrolidone, gelatin and alginates. Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The peptides of the present invention may be used as separately administered compositions or in conjunction with other agents.

The peptides of this invention can be lyophilised for storage and reconstituted in a suitable carrier prior to use. This technique has been shown to be effective and art-known lyophilisation and reconstitution techniques can be employed. It will be appreciated by those skilled in the art that lyophilisation and reconstitution can lead to varying degrees of activity loss and that use levels may have to be adjusted upward to compensate.

### Examples

The following examples are intended to illustrate aspects of the claimed invention, but not be limiting in any manner, unless explicitly recited as a limitation.

### Methods

### Patient Sample Collection

Human lens capsules were collected from patients having an age range of 63-84 years, mean 74.8±5 years, undergoing phacoemulsification cataract surgery, and was stored in the balanced salt solution (BSS® intraocular irrigating solution, Alcon) at 4° C. prior to use. Aqueous humor fluid was collected from the anterior chamber of the eye using a 30-gauge cannula inserted through the paracentesis site. Patients with a history of diabetes mellitus, with previous severe trauma to the eye, with previous expositor to infrared radiation and patents with the previous diagnosis of amyloid disease were excluded from the study.

### Cell Line

Primary human trabecular meshwork (hTM) cells were obtained from ScienCell Research Laboratories (Carlsbad, CA), and maintained in TMCM medium (ScienCell, no. 6591). Primary cell culture was passaged according to the manufacturer's instructions. Passage three cells were seeded on tissue culture plates coated with gelatin and media were refreshed every 2-3 days. These monolayer cultures were used in subsequent experiments upon reaching 95-100% confluency.

### Isolation of XFS Material-Specific Peptides

Peptide sequences that bind to XFS materials were deduced from the DNA sequences of selected phage clones. All phage binding was verified using extracted human aqueous humor (hAH) so as to mimic the physiological pH and solution properties that are crucial to molecular interactions (i.e., ion, protein, osmolarity) [25].

Ph.D.™-12 phage display peptide library was used for ex vivo screening. All human tissues were washed three times with BSS buffer before use. Human lens capsules collected from patients without XFS were used for subtractive screening. The lens capsules were incubated with the phage library (1·10¹¹ pfu) in an equal volume of aqueous humor fluid and BSS® irrigating solution for 1 hr at 37° C. in 0.2 ml tube. The solution was removed and 300 μl of ice-cold BSS solution was added to the tube and tissue was washed several times with BSST buffer (BSS solution containing 0.1% v/v Tween-20) to elute off unbound or weakly bound phages. The lens capsule was subsequently stained with 0.06% trypan blue (same concentration used in anterior segment surgeries to facilitate visualization of target tissues in specific situations) to have a better visualization of XFS materials under the microscope. Tissue was washed further to remove the excess dye and was placed on a sterile microscope slide and covered with 20 μl of BSS buffer and XFS materials were carefully removed from the surface of the lens capsule using gel-loading pipette tips (GELoader, epT.IPS, 20 μL, Eppendorf, Germany). Care was taken to not remove undesired parts of the lens capsule during the process. The solution containing the isolated XFS materials was transferred to a fresh tube containing 100 μl 0.2 M glycine-HCl (pH 2.2) to elute phages bound to the XFS materials. After 15 min the solution containing recovered phages was neutralized with 15 μl 1 M Tris-HCl buffer (pH 9.1). The eluted phages were amplified by infection of E. coli host strain ER2738 (New England Biolabs). Three rounds of ex vivo panning were carried out with stepwise increasing of Tween concentration in BSST buffer (0.1, 0.2, 0.3%) to increase the likelihood of identification of XFS materials-targeting peptides. Individual clones were then picked for the characterization of peptide-encoding inserts using DNA sequencing. 12 clones were picked from the first and second round each and analyzed via DNA sequencing to make sure that there was no growth advantage happening over the library at the beginning of the screening. 52 clones were subsequently picked from the ex vivo-identified XFS-targeting phages and their peptide-encoding DNA inserts were analyzed using DNA sequencing.

Three rounds of biopanning against XFS materials sourced from different patients yielded an enrichment of two peptides: LPSYNLHPHVPP (p-LPS) [SEQ ID NO. 1] and IPLLNPGSMQLS (p-IPL) [SEQ ID NO. 2]. The high degree of selectivity of these phage towards XFS materials was aided by: i. negative screening through removal of phage that bound normal lenses through negative biopanning, and ii. removal of XFS materials from the lens capsules, resulting in specific amplification of phage that only bound to these fibrils. Moreover, the robust nature of the binding was evident as samples came from a host of different patients yet the responses were similar. Finally, the use of extracted human aqueous humor at 37° C. [26] was vital to these experiments so as to maintain a solution environment (i.e., pH, ionic strength, proteins, osmolarity) that was as close as possible to the physiological environment where the targeting of fibrils would occur.

### Evaluating the Targeting Ability of Phage-Displayed Peptides

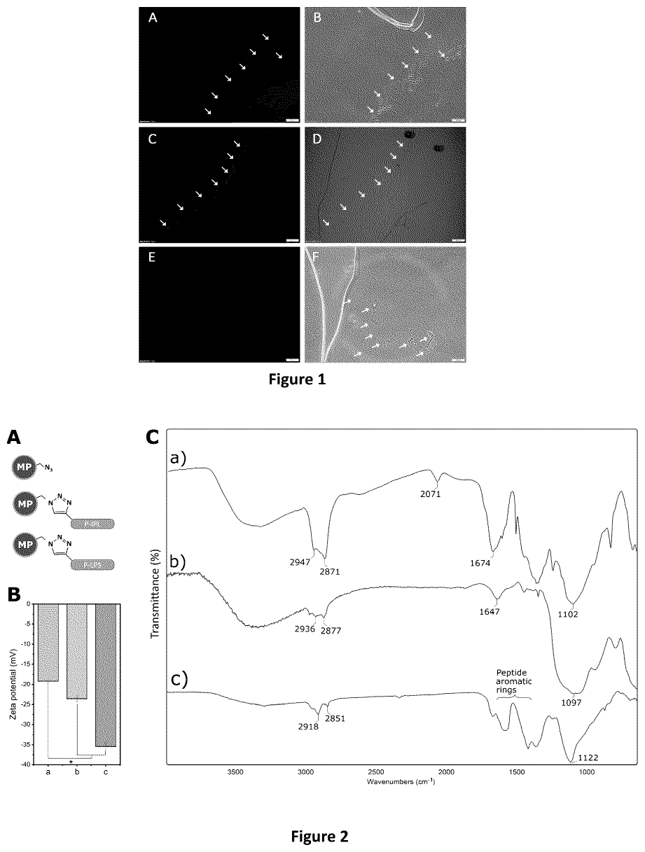

Human lens capsules with XFS materials were stained with fluorescently labeled wild-type phages as well as phages displaying the enriched peptides. It is important to note that the conjugated fluorophore (Cy5 NHS ester dye) molecule did not interfere with phage binding to the XFS materials as it reacts with the primary amino group of lysine, which was not present in the enriched peptides. Both phage-displayed peptides (p-LPS and p-IPL) showed specific binding to XFS materials (FIG. 1A, 1C), where the presence of XFS materials was already confirmed using bright-field microscopy (FIG. 1B, 1D, 1F). Wild-type phages showed no noticeable interaction with XFS materials on the surface of the lens capsule (FIG. 1E). XFS materials do not cover the whole surface of the lens cuspule, meaning that labeled phages had the chance to interact with the non-XFS altered regions of the lens capsule. Phage were observed to bind only to the XFS regions of the lens capsule, further confirming their specificity towards the XFS materials.

### Phage Labeling

The ability of two highly enriched phage-displayed peptides to bind specifically to XFS materials was evaluated ex vivo by fluorescently labeling these phages with Cy5 (Lumiprobe) as described elsewhere [40]. Amplified phages (1×10¹¹ pfu) were resuspended in 0.3 M NaHCO₃ (pH 8.6) containing 10 μg Cy5 dye and incubated for 2 hr at room temperature in the dark. Subsequent to phage/fluorophore incubation, 40 μl of 10 mM lysine was added to interact with remaining free Cy5 dye molecules in the solution. The volume of the reaction mixture was subsequently brought up to 1 ml with PBS buffer, and the phages were purified with two rounds of 20% (w/v) polyethylene glycol-8000, 2.5 M NaCl precipitation. The labeled phages resuspended in BSS solution.

### Ex Vivo Evaluation of Targeting Ability of Labeled Phages

Fluorescently labeled phages carrying identified targeting peptides as well as wild-type phages without peptide-encoding inserts were incubated with exfoliative human lens capsules in a 100-μl solution containing equal volumes of human aqueous humor fluid and BSS solution. The incubation was allowed to continue for 1 hr at 37° C. After serially washing with BSST buffer (0.1, 0.3%), three times each, the lens capsules were mounted on microscope slides and were examined under an Olympus IX81 inverted fluorescence microscope (Olympus Corporation, Tokyo, Japan). The location of XFS materials on the surface of the lens capsule was confirmed in bright-field mode prior to fluorescence imaging.

### Magnetic Bead-Peptide Conjugates

The targeting capability of peptide modified magnetic particles (MPs) was confirmed against their scrambled sequences for binding to human lens capsules with and without XFS materials. Targeting capability of MP-peptide complexes to XFS materials was studied ex vivo in the same experimental conditions that phage panning was conducted. Cellular uptake of MP-peptide conjugates was studied using electron microscopy. Cytotoxicity of MP-peptide complexes was evaluated using live/dead cell viability assay, MTT assay, and DNA fragmentation. The effect of a magnetized pin or a rotating magnetic field on the removal of XFS materials bound to peptide modified MPs was evaluated using XFS lens capsules (ex vivo).

Azide-functionalized iron oxide core magnetic particles with biocompatible coatings and having a diameter of 1 μm (purchased form Nanocs Inc, New York) were used in this study. Synthetic alkyne-modified peptides (>95% purity) corresponding to the phage-displayed XFS materials-binding peptides and scrambled sequences were purchased from RS synthesis (Louisville, KY, USA). The peptides were alkyne modified and their conjugation to azide-functionalized MPs were carried out through copper-catalyzed azide-alkyne click chemistry as described Copper-Catalyzed Azide-Alkyne Click Chemistry for Bioconjugation [41].

Azide-functionalized MPs were added to the peptide solution having a final concentration of 600 μM in 100 mM potassium phosphate buffer (pH 7). A premix solution containing 2.5 μl of 20 mM CuSO₄ and 5 μl of 50 mM tris(3-hydroxypropyltriazolyl-methyl)amine (THPTA) ligand (Lumiprobe) was prepared immediately prior to use and added to the click reaction solution. 25 μl of 100 mM sodium ascorbate was subsequently added and the reaction was allowed to proceed for 1 hr. Subsequent to click reaction, the peptide-conjugated MPs were first washed with 10 mM EDTA to remove copper ions and then with BSS buffer.

### Characterization of Peptide-Conjugated MPs

The surface charge of MPs with and without peptide conjugates was measured using a Zetasizer Nano ZS (Malvern Instruments, UK). For FTIR measurements, drop-cast films of

MPs with and without peptide conjugates were analyzed using an FTIR microscope (Nicolet continuum FTIR microscope (Thermo Scientific). FTIR spectra were collected with a resolution of 4 cm⁻¹ and 128 scans of each sample. Conjugation of peptides to MPs was further analyzed through competitive labeling of MPs with 5-carboxytetramethylrhodamine alkyne (TAMRA-alkyne), 5-isomer fluorophore (Lumiprobe). Azide-functionalized MPs were conjugated first with targeting peptides and then labeled with TAMRA-alkyne fluorophore as described before in the conjugation section. Since the azide groups on the surface of MPs had already interacted with the alkyne group of peptides, they were expected to be non- (or less-) labeled compared to control particles (without peptide conjugates).

### Peptide-Particle Conjugation

Conjugation of alkyne-modified peptides to azide-functionalized MPs (FIG. 2A) was confirmed with surface zeta potential measurements, Fourier-transform infrared spectroscopy (FTIR), and a competitive inhibition assay. Peptide tethering through the N-terminal domain was expected to yield an increase in negative charge on the surface of the MPs, as was observed (FIG. 2B). FTIR spectrum from MPs before conjugation to peptides showed a transmittance peak around 2071 cm⁻¹, which is attributed to the asymmetric stretching vibration of the free azide groups (FIG. 2C. a). The free azide group was absent in the spectra of peptide-conjugated MP samples due to the conversion of free azide groups to triazole ring during azide-alkyne cycloaddition (FIG. 2C. b, c). The peak observed at 1647 cm⁻¹ in p-IPL could represent carbonyl groups of amide bonds of the peptide (FIG. 2C. b). The bands at wavelengths between 1400-1650 cm⁻¹ correspond to aromatic rings found on p-LPS. (FIG. 2C. c).

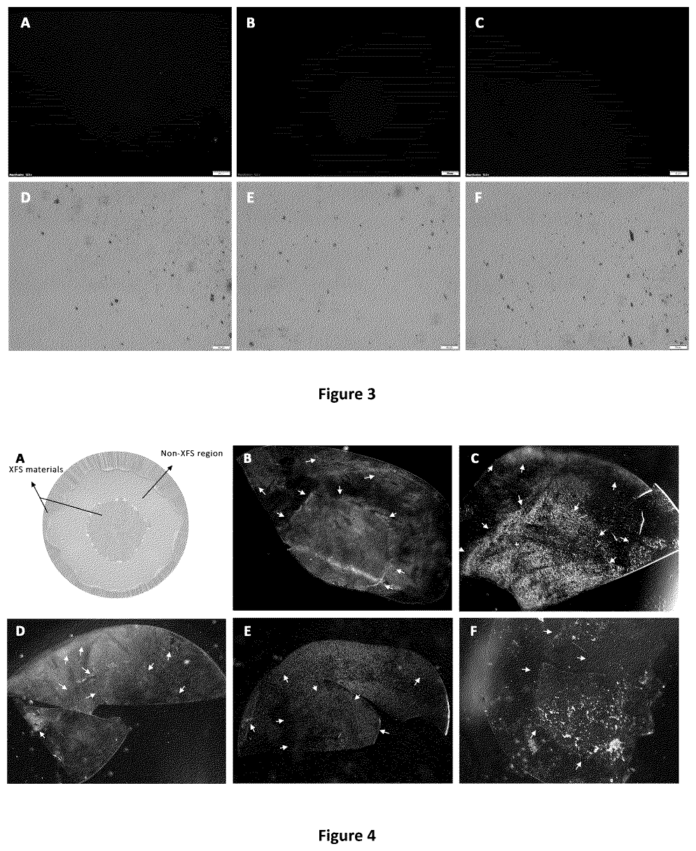

Conjugation of peptides to MPs was confirmed by labeling particles with or without conjugated peptides with an azide-reactive dye (TAMRA alkyne fluorophore), where subsequent fluorescence indicated that unreacted azides were present on the MP surface (FIG. 3). MPs were reacted with p-IPL (FIG. 3B) or p-LPS peptides (FIG. 3C), those MPs had no fluorescence compared to the control MPs that had no conjugated peptides (FIG. 3). Given the excessive amount of peptides used to react with available azides on the magnetic particles, coupled with the lack of any unreacted azides (FIG. 3B and 3C), it is reasonable to conclude that nearly 100% of all azides reacted to covalently tether peptides to the magnetic particles.

### Ex Vivo Evaluation of Targeting Ability of Identified Peptides

Human lens capsules obtained from XFS patients after washing with BSS buffer were transferred into a solution containing equal volumes of human aqueous humor and BSS irrigating solution. 25 μg of each control MPs (without peptide conjugates), MP-peptide, and scrambled peptide-MP complexes were incubated with lens capsules in that solution in a 0.2 ml tube for 1 hr at 37° C. with gentle shaking. Afterward, the excess particles were washed off with BSS buffer and the lens capsules were mounted on the microscope slide and images were taken using an Axiocam-105 color camera on a stereomicroscope (Stemi-305, Carl Zeiss).

### Targeting Capability of MP-Peptide Conjugates

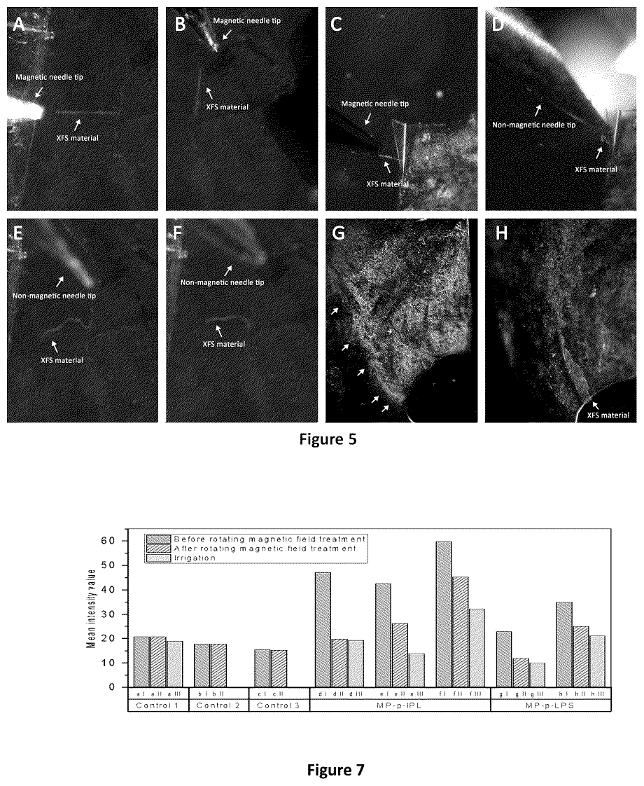

It is important to note that although there might be patient-to-patient variations in the XFS deposition pattern on the lens capsule, generally it is distributed with a non-XFS intermediate zone that separates exfoliative central and peripheral zones (FIG. 4A) [11]. Specific targeting of XFS materials was evaluated through incubating MP-p-IPL, MP-p-LPS, MP-scrambled peptides (control), or unmodified MP (control) with XFS affected lens capsules in equal volume of extracted aqueous humor fluid and BSS irrigating solution at 37° C. Specific binding of MP-p-IPL and MP-p-LPS to XFS materials was observed (FIG. 4B, C). This confirms that MP-peptide conjugates resulted in similar binding patterns as that observed for just fluorescently labeled phage-displayed peptides (FIG. 1). Whereas, scrambled peptide complexes showed a non-specific binding and the whole surface of the lens capsule having was covered with MPs regardless of XFS presence (FIG. 4D, E). The other control experiment using virgin MPs resulted in large clumps of MPs on the central zone (FIG. 4F).

### Evaluation of Behavior of Magnetized XFS Materials Under Magnetic Field

### Rotating Magnetic Field Studies

In order to better evaluate of the effect of rotating magnetic field on removing of XFS materials from the surface of lens capsules, images of lens capsules which were captured before and after treatment with the rotating Halbach array were converted to 16-bit gray-scale images and their intensities analyzed using Adobe Photoshop CC 2015 software (Adobe Systems Inc, San Jose, CA).

The same approach was followed as previously described except for using a rotating magnetic field instead of the static magnetic field applied with a magnetic pin. The lens capsules were first incubated with MP-peptide conjugates and then treated with a rotating Halbach array magnet that produced a ˜5000 G magnetic field across the gap of the magnet (Nickel-plated N48H, Super Magnet Man Inc., Alabama, USA). The tissue samples were incubated at the entrance of the Halbach array gap rotating at 20 Hz for 3 hr and then images were taken under the stereomicroscope. Irrigation and aspiration is used in cataract surgery to remove remaining parts of the lens materials and residual viscoelastic solution from the eye. To mimic the surgery conditions, irrigation of BSS buffer at a rate of 10 ml/min was applied over the processed tissues to observe the effect of buffer irrigation on the removal of the XFS materials after being treated with rotating magnetic field. Control studies were carried out with the same treatment approaches using XFS lens capsules, except for using BSS buffer instead of MP-peptide conjugates.

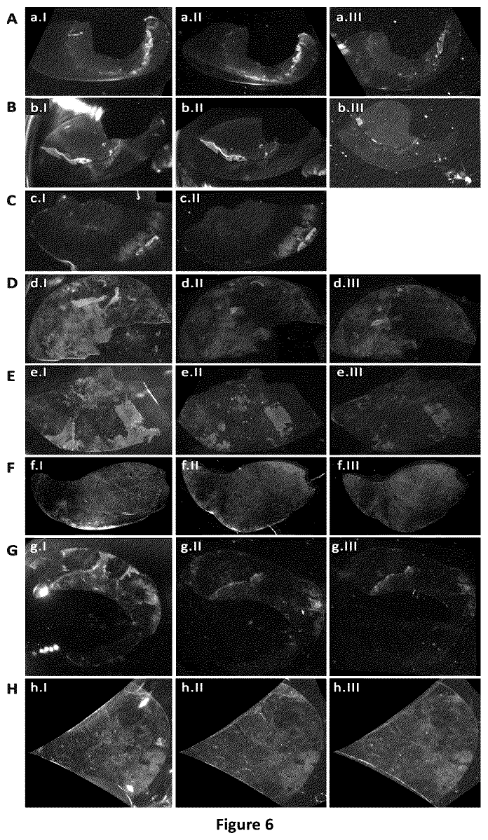

Images of tissues before and after applied magnetic field and irrigation (FIG. 6), were analyzed based on the intensity of the lens capsule surface before and after each test (FIG. 7).

A rotating Halbach array was used to induce an external magnetic field on XFS laden lens capsules incubated with peptide-decorated MPs. It was observed that the field strength generated was sufficient enough to lead to the removal of a significant amount of XFS materials from the surface of the lens capsule (FIG. 6). Furthermore, the effect of irrigation after agitation by MPs under the magnetic field was used to evaluate the removal of these materials. It was observed that irrigation after magnetic field treatment lead to a further significant removal of XFS materials as compared to irrigation or MP treatment alone. As an example, a lens capsule with central zone deposits that was bound to MP-p-IPL particles (FIG. 6D) showed a large amount of XFS removal upon applying the rotating magnetic field (FIG. 6DII). Another tissue having XFS deposits on both central and peripheral zones (FIG. 6E), bound with MP-p-IPL particles, showed that the application of the rotating magnetic field removed aggregates from both zones of the tissue (FIG. 6EII). MP-p-IPL bound materials on a lens capsule with XFS deposits on the central zone had all large XFS aggregates removed only through the magnetic field (FIG. 6F). However, this tissue had a dense amount of cataractous materials on the posterior side of the lens capsule, which caused the attachment of MPs to those materials making the evaluation difficult. Therefore, although not as visually effective as the previous tissues, the rotating magnetic field was still actually effective in the removal of the large XFS aggregates from the surface of that lens capsule (FIG. 6FII). Lens capsules shown in (FIG. 6G and H) were exposed to MP-p-LPS conjugates and treated with the rotating magnetic field. The central zone of one sample (FIG. 6G) was lost due to surgery, however, XFS materials on the lens surface were mostly removed after applying the rotating magnetic field (FIG. 6GII). The rotating field was also effective in removing some of XFS aggregates from the other tissue (FIG. 6H) incubated with MP-p-LPS. In this case, the peripheral zone of the tissue was mostly cut out of the images due to the coverslips used for holding the tissue in place during irrigation. Irrigation of BSS buffer over the tissue was shown to be effective in removing some XFS aggregates from the surface of the lens capsule (FIG. 6D-H). The control tissues that were treated with the rotating magnetic field showed almost no removal of XFS materials (FIG. 6A-C). Due to the tissue handing difficulties, we could not do BSS irrigation on one of the control tissues (FIG. 6C). XFS materials being partially lost in the control lens capsule shown in (FIG. 6BIII) was not due to the effect of magnetic field, but rather to sample handing being responsible for the loss of the fragile materials that were already lifted from the lens capsule. The control lens capsule shown in (FIG. 6A) showed a very little removal of XFS aggregates during BSS irrigation and no effect was observed after treating with rotating magnetic field (FIG. 6AIII).

Results demonstrate that irrigation after MP treatment leads to enhanced removal of XFS materials from the surface of ex vivo lens capsules. Upon removal of XFS materials, the commonly used irrigation/aspiration system can remove large and small XFS materials from the anterior chamber of the eye. It is thought that due to the targeting ability of our designed MP-peptide system against XFS materials, this technique has the potential to eliminate XFS materials from the majority of surfaces in the anterior ocular chamber.

### Magnetic Pin Test Effect of Magnetic Field on XFS Materials

Micron-sized, iron oxide particles were used as they are clinically approved for biomedical applications, less susceptible to nonspecific cellular uptake or vascular egress, rapidly cleared (<5 min) by the liver and spleen, and have a high labeling valency that enhances their binding affinity to molecular targets [21,27-30]. These particles are biodegradable, where particles (>150 nm) are captured by phagocytic cells and their coating cleaved by lysosomal enzymes, and the iron oxide core is degraded into iron and oxygen through mechanisms involved in iron metabolism [31,32].

A magnetized pin was used to demonstrate that MP-bound XFS deposits could be affected via a localized magnetic field. The magnetic force generated at the tip of the pin was not strong enough to remove MP-XFS aggregates, however, it was able to re-orient them in the direction of the applied field (FIG. 5A-C); contrary to a non-magnetic needle control (FIG. 5D-F). For some lens capsules, these magnetic pins did start to pull the edges of XFS materials off the lens capsule, but was unable to remove these deposits entirely (FIG. 5G, H). However, any XFS materials that were already detached and in solution due to irrigation or surgical manipulation could be collected using a magnetized pin.

Human lens capsules incubated with MP-peptide complexes were laid flat on a microscope slide with the XFS side facing up and 50 μl of BSS buffer was placed on the top of the tissues. A metallic pin which was magnetized by attaching it to the surface of a permanent magnet having field strength at the pole of ˜5000 G was used to observe the effect of applied magnetic tool on the XFS materials. Control experiments were carried out on the same tissues using a non-magnetic 23-gauge needle which had almost the same tip size as previously used magnetic pins. Images were taken under the stereomicroscope.

The effect of the magnetic pin and rotating magnetic field on the behavior of magnetized XFS materials was observed under the stereomicroscope. A low frequency rotating magnetic field which was producing a uniform magnetic field across the designed system provided suitable conditions to remove most of the XFS aggregates from the surface of lens capsules. Compared to non-XFS cases, XFS patients show greater IOP lowering effect following phacoemulsification cataract extraction, where this IOP decrease was found to be proportional to irrigation volume used during cataract surgery [36]. This has been speculated that washing out of XFS materials could be one of the reasons for observing greater IOP drop in XFS patients [36,37].

### Cytotoxicity Studies of MP-Peptide Conjugates

Prior to toxicity studies, the common phenotypic features of cultured hTM cells were assessed using immunohistochemical studies. The population doubling time for obtained hTM cells was determined to be 11.3±2.1 hours, which is typical for fetal hTM cells. Immunohistochemical analyses of hTM cells included evaluation of expression of fibronectin, myocilin, laminin, and actin. Is has been shown that actin microfilaments are mainly aligned parallel to the longitudinal axis of cultured hTM cells, which was also observed for our cells when stained with phalloidin dye. Expression of fibronectin protein was also confirmed in the cultured hTM cells. This protein is one of the major extracellular matrix glycoproteins of hTM cells which is secreted as loosely aligned filaments on the cultured hTM cells surface or large bundles at the periphery of individual cells. Myocilin protein (or trabecular meshwork-inducible glucocorticoid response protein) is a highly expressed glycoprotein in the trabecular meshwork, and has been found within the cytoplasm of hTM cells when in association with extracellular matrix components. The expression of myocilin is a key phenotypic characteristic of hTM cells and was observed in our cell culture. Expression of laminin is a marker to characterize cultured hTM cells, and was observed in immunohistochemical studies. The common morphology of hTM cells is the spindle-like shape which was also observed in monolayers of cultured hTM cells.

The in vitro cytotoxicity of peptides in free and MP-conjugated form was evaluated against obtained hTM cells using live/dead cell viability assay, MTT assay, and DNA cleavage. All in vitro cell assays showed that both free peptide and MP-peptide particles were not toxic in the concentrations studied, as detailed below.

### Electron Microscopy Studies

### Scanning Electron Microscopy (SEM)

The monolayer of hTM cells cultured on gelatin-coated glass coverslips in 24-well plates were incubated with 100 μg of MP-peptide complexes for 2 hr at 37° C. The wells were topped up with the fixative solution (2.5% glutaraldehyde, 4% paraformaldehyde in 0.1 M phosphate buffer, pH 7.4), for 20 min. Upon serial washing with PBS buffer (pH 7.4), samples were dehydrated with graded ethanol gradually by 20% increments for 30 min each until 100% ethanol. Samples were subsequently treated with hexamethyldisilazane (HMDS) and allowed to dry overnight, then mounted on SEM stubs and sputtered with a gold-palladium film. SEM images were obtained using a Zeiss Sigma FE-SEM scanning electron microscope (Carl Zeiss, Inc., Oberkochen, Germany) at the accelerating voltage of 20 kV.

### Transmission Electron Microscopy (TEM)

Confluent monolayers of hTM cells cultured in 24-well cell culture plates were incubated with MP-peptide complexes as previously described for SEM studies. After washing with Dulbecco's PBS buffer, cells were scraped off the wells and transferred into the microcentrifuge tube. Cells were centrifuged at 1000 rpm for 5 min to form a pellet. The supernatant was removed and the cells pellet was incubated in the fixative solution overnight. Upon washing with 0.1 M phosphate buffer (pH 7.4), samples were post-fixed in 1% osmium tetroxide for 1 hr. Dehydrated pellets were then embedded in Spurr's resin and allowed to polymerize at 70° C. overnight. Ultrathin sections were prepared using an ultramicrotome (Reichert-Jung Ultracut-E, Vienna, Austria) with a diamond knife. The ultrathin sections were stained with 4% uranyl acetate solution and imaged using a Philips-FEI, Morgagni-268 transmission electron microscope (Hillsboro, USA) operating at an acceleration voltage of 80 kV.

### Morphology and Phenotype of hTM Cells

Before biocompatibility studies, the morphology and phenotypic characteristics of cultured hTM cells were studied. Population doubling time of hTM cells was obtained as described elsewhere [42].

### Immunohistochemical Study

Immunohistochemical assays were employed to identify the expression of characteristic phenotypic markers of trabecular meshwork cells. Confluent monolayers were confirmed using an inverted cell culture microscope (Leica DMi1, Leica Microsystems Inc,

Germany). Immunohistochemical labeling was conducted according to the manufacturer's protocol. Rabbit anti-fibronectin (1:200, ab2413, Abcam), rabbit anti-laminin (1:200, ab 11575, Abcam) and rabbit anti-myocilin (1:200, ab41552, Abcam) antibodies were used as fluorescently-labeled primary antibodies. Phalloidin-Alexa Fluor 488 (1:40, Molecular Probes) was used to label filamentous actin (F-actin). Upon washing with PBS buffer, grown cells on glass coverslips were fixed with 4% paraformaldehyde in PBS buffer for 10 min at room temperature. Cells were subsequently washed with ice-cold PBS buffer and permeabilized for 5 min with 0.2% Triton X-100. Cells were washed thrice with PBS buffer and incubated with PBS containing 5% goat serum for 1 hr at room temperature to block nonspecific binding sites of the antibodies. Primary antibodies were then added to the wells ad incubated overnight at 4° C. in the dark. Upon washing with PBS buffer, cells were incubated with phalloidin for 20 min. Samples were then covered using fluoroshield mounting medium with DAPI (ab104139, Abcam) and images were taken using an Olympus IX81 inverted fluorescence microscope (Olympus Corporation, Tokyo, Japan).

### Biocompatibility Studies

Biocompatibility of free peptides and corresponding MP conjugates was confirmed using MTT cell toxicity assay and live/dead cell proliferation assay. DNA fragmentation studies also showed that either MP-peptide conjugates or free peptide solutions did not induce apoptotic cellular death in hTM cells. Electron microscopy studies showed that compared to the particles being in aggregated form, individual particles were more likely to be taken up by the cultured hTM cells.

### MTT Cytotoxicity Assay

Cytotoxicity of XFS-targeting peptide solutions, as well as MP-peptide conjugates, was tested against hTM cells using MTT assay. Cells were plated in 24-well plates and upon reaching confluency they were treated with different concentrations of peptide solutions and particle-peptide conjugates. Cell proliferation was evaluated 24 hr post-treatment using the Vybrant® MTT cell proliferation assay kit (Molecular Probes).

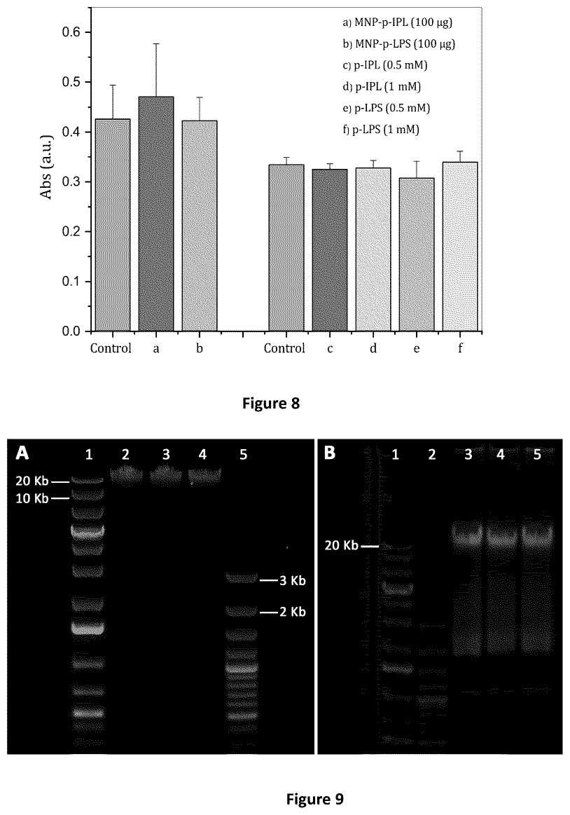

hTM cell proliferation in the presence of solution free and MP bound peptides was conducted using the MTT colorimetric assay. A one-way ANOVA test showed that no significant difference (p<0.05) was observed between the absorbance of hTM cells treated with MP-peptide conjugates or solution free peptides compared to control cells treated with water (FIG. 8). The results confirmed that both sets of test samples did not suppress the proliferation of hTM cells.

### Live/Dead Cell Viability Assay

Cell viability was further assayed using LIVE/DEAD® viability/cytotoxicity kit (Molecular Probes). Monolayers of hTM cells were treated in triplicates with peptides alone in solution (1 mM) and in conjugation with MPs (100 μg). Cell viability was then assessed according to the manufacturer's protocol. The kit is based on calcein AM/ethidium homodimer-1 (EthD-1) system, where calcein AM detects cellular esterase activity of live cells and EthD-1 stains nuclei of dead cells. A similar live/dead assay was used to investigate the effect of MP-peptide conjugates on the viability of hTM cells in the presence of the rotating magnetic field. Cells grown in 24-well plate and treated with MP-peptide conjugates were placed under a rotating magnetic field for 1 hr. The viability of hTM cells was then assessed as described above. A similar live/dead assay was carried out to evaluate the effect of the rotating magnetic field on cultured hTM cells. The plated cells in a 24-well cell culture plate were incubated with 100 μμg MP-peptide conjugates. The plate was placed under a rotating magnetic field which was constructed using two attached cuboid-shaped permanent magnet (having a pole field strength of ˜5000 G) to provide a larger field over the plate. The cells were treated with the rotating magnetic field for 10 min and then analyzed using live/dead assay as described before. For both MTT and live/dead assays one-way ANOVA test was used to analyse the significance of difference (p<0.05) between the results.

Live/Dead cell Viability Assay Performed on hTM Monolayers

XFS-specific peptides in both solution free or MP bound form were incubated with hTM cells and their cytotoxic effects assessed. It was found that both MP-p-IPL and MP-p-LPS constructs were not toxic in the concentrations studied. Confluent cultured cells were incubated with 50 μg and 100 μg of MP-p-IPL or MP-p-LPS. hTM monolayers were also incubated with 1 mM of p-IPL or p-LPS peptide solutions, and their viability was analyzed in a similar way as the MP-peptide constructs. As seen in the case of MP-peptide conjugates, free peptides were not toxic in used concentrations (Table 1). Live/dead assay of hTM cells treated with rotating magnetic field also showed that cells incubated with MP-peptide conjugates did not show significant loss compared to control cells incubated with water (Table 2). Because of the sensitivity of hTM cells, it was not possible to treat cells with a rotating magnetic field outside of a CO₂ regulated atmosphere for longer than 10 min. That said, even control cells showed significant loss upon removal from the CO₂ incubator, thus, an optimal 10 min exposure to the magnetic field was chosen to evaluate the viability of cells in the presence of rotating magnetic field (Table 2).

Table 1. Live/dead assay performed on hTM monolayers treated with MP-peptide conjugates and free peptide solution. No statistical significant difference (P<0.05) was found between studied groups. (Data represent mean±1 SD, n≥3).

| Peptide-particle conjugates | | Free peptide solution | |
|---|---|---|---|
| Sample | % of live cells | Sample | % of live cells |
| Control | 99.01 ± 0.51 | Control | 99.36 ± 0.36 |
| MP-p-ILP (50 μg) | 99.60 ± 0.18 | 1 mM p-IPL | 99.00 ± 0.37 |
| MP-p-ILP (100 μg) | 99.66 ± 0.07 | 1 mM p-LPS | 99.12 ± 0.94 |
| MP-p-LPS (50 μg) | 99.58 ± 0.20 | | |
| MP-p-LPS (100 μg) | 98.47 ± 1.46 | | |

Table 2. Live/dead assay performed on hTM monolayers incubated with MP-peptide conjugates and treated under 10 min rotating magnetic field. No statistical significant 10 difference (P<0.05) was found between studied groups. (Data represent mean±1 SD, n≥3).

Peptide-particle conjugates

| Sample | % of live cells |
|---|---|
| Control | 98.74 ± 0.45 |
| MP-p-ILP (100 μg) | 98.53 ± 1.10 |
| MP-p-LPS (100 μg) | 98.88 ± 0.51 |

### DNA Fragmentation Analysis

Effect of free and conjugated XFS-targeting peptides on the induction of apoptosis was investigated via chromosomal DNA fragmentation analysis: DNA fragments indicate apoptosis induction. MP-peptide constructs, as well as solution free peptides, did not initiate apoptosis for hTM cells; chromosomal DNA remained intact after 24 hr incubation with MP-p-IPL, MP-p-LPS, p-LPS, or p-IPL solutions (FIG. 9).

Effect of peptide-particle conjugates and peptide solutions on the induction of apoptotic DNA fragmentation was analyzed in hTM cells. Grown hTM cells in 24-well plates were incubated with 100 μg of peptide-MP complexes or 0.5 mM peptide solutions for 24 hr at 37° C. Cells were recovered from wells using a cell scraper. DNA was isolated and purified using DNeasy blood and tissue kit (Qiagen). Extracted DNA molecules from control and treated hTM cells were subjected to 2% agarose gel electrophoresis and visualized by SYBR green staining.

Table 3. Peptide enrichment during ex vivo biopanning against human lens capsules.

| | | | % of | |
|---|---|---|---|---|
| Biopanning | Peptide | copies | sequences | Lens capsule type |
| Negative | — | — | — | without XFS |
| panning | | | | materials |
| (subtraction) | | | | |
| Round 1 | — | — | — | with XFS materials |
| Round 2 | p-LPS | 3/12 | 25 | with XFS materials |
| | p-IPL | 4/12 | 33 | |
| Round 3 | p-LPS | 11/52 | 21 | with XFS materials |
| | p-IPL | 37/52 | 71 | |

### Cell Uptake Studies Using Electron Microscopy

One of the key functions of trabecular cells is the phagocytosis of foreign materials and extracellular debris [33], a cellular function also observed in vitro [34]. The internalization of MP-peptide constructs into hTM cells may lead to cell death if exposed to an oscillating magnetic field. Thus, hTM cells were incubated for 2 hr with 100 μg of each of MP-p-IPL or MP-p-LPS and TEM micrographs showed that individual particles were taken up by hTM cells, and some particles were engulfed by hTM cells. Aggregates of both MP-p-IPL and MP-p-LPS particles were observed outside of cultured hTM cells as well. Individual particles, as well as aggregated MPs, were also observed in SEM micrographs of regions surrounding cultured hTM cells. It seems that MPs in large aggregates have less propensity to enter the cultured cells. It has been previously shown that intravitreal or anterior chamber injection of magnetic nano-and microparticles had almost no signs of effect on retinal morphology, photoreceptor function or IOP in the anterior chamber of animal models [35]. Regardless, according to different cell viability assays conducted in this study, no statistical difference was observed for cell mortality upon incubation with MPs in cultured hTM cells.

### Definitions and Interpretation

The description of the present invention has been presented for purposes of illustration and description, but it is not intended to be exhaustive or limited to the invention in the form disclosed. Many modifications and variations will be apparent to those of ordinary skill in the art without departing from the scope and spirit of the invention. Embodiments were chosen and described in order to best explain the principles of the invention and the practical application, and to enable others of ordinary skill in the art to understand the invention for various embodiments with various modifications as are suited to the particular use contemplated.

The corresponding structures, materials, acts, and equivalents of all means or steps plus function elements in the claims appended to this specification are intended to include any structure, material, or act for performing the function in combination with other claimed elements as specifically claimed.

References in the specification to “one embodiment”, “an embodiment”, etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to affect or connect such aspect, feature, structure, or characteristic with other embodiments, whether or not explicitly described. In other words, any element or feature may be combined with any other element or feature in different embodiments, unless there is an obvious or inherent incompatibility between the two, or it is specifically excluded.

It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an item, condition or step being referred to is an optional (not required) feature of the invention.

The singular forms “a,” “an,” and “the” include the plural reference unless the context clearly dictates otherwise. The term “and/or” means any one of the items, any combination of the items, or all of the items with which this term is associated.

As will be understood by the skilled artisan, all numbers, including those expressing quantities of reagents or ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values and ranges proximate to the recited range that are equivalent in terms of the functionality of the composition, or the embodiment.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, as used in an explicit negative limitation.

### References

The following references are indicative the level of skill in the art, and are incorporated by reference herein in their entirety, where permitted. Any inconsistency with the present application may be interpreted as an alternative or optional interpretation, statement or element.
1. Ritch R. Exfoliation syndrome-the most common identifiable cause of open-angle glaucoma. J Glaucoma. United States; 1994;3:176-7.
2. Davanger M. The pseudo-exfoliation syndrome. A scanning electron microscopic study. I. The anterior lens surface. Acta Ophthalmol. Denmark; 1975;53:809-20.
3. Ghosh M, Speakman JS. The ciliary body in senile exfoliation of the lens. Can J Ophthalmol. England; 1973;8:394-403.
4. Harnisch JP. Exfoliation material in different sections of the eye. Albrecht Von Graefes Arch Klin Exp Ophthalmol. Germany; 1977;203:181-90.
5. Schlötzer-Schrehardt U, Küchle M, Naumann GO. Electron-microscopic identification of pseudoexfoliation material in extrabulbar tissue. Arch Ophthalmol. 1991;109:565-70.
6. Streeten BW, Gibson SA, Dark AJ. Pseudoexfoliative material contains an elastic microfibrillar-associated glycoprotein. Trans Am Ophthalmol Soc. United States; 1986;84:304-20.
7. Ritch R, Schlötzer-Schrehardt U, Konstas AGP. Why is glaucoma associated with exfoliation syndrome? Prog Retin Eye Res. 2003;22:253-75.
8. Hollo G, Katsanos A, Konstas AG. Management of exfoliative glaucoma: challenges and solutions. Clin Ophthalmol. New Zealand; 2015;9:907-19.
9. Aboobakar IF, Johnson WM, Stamer WD, Hauser MA, Allingham RR. Major review: Exfoliation syndrome; advances in disease genetics, molecular biology, and epidemiology. Exp Eye Res. Elsevier Ltd; 2017;154:88-103.
10. Ritch R. Ocular and systemic manifestations of exfoliation syndrome. J Glaucoma. 2014;23: S1-8.
11. Ritch R, Schlötzer-Schrehardt U. Exfoliation syndrome. Surv Ophthalmol. 2001;45:265-315
12. Elhawy E, Kamthan G, Dong CQ, Danias J. Pseudoexfoliation syndrome, a systemic disorder with ocular manifestations. Hum Genomics. England; 2012;6:22.
13. Citirik M, Acaroglu G, Batman C, Yildiran L, Zilelioglu O. A possible link between the pseudoexfoliation syndrome and coronary artery disease. Eye (Lond). England; 2007;21:11-5.
14. Gonen KA, Gonen T, Gumus B. Renal artery stenosis and abdominal aorta aneurysm in patients with pseudoexfoliation syndrome. Eye (Lond). England; 2013;27:735-41.
15. Ma Z, Qin H, Chen H, Yang H, Xu J, Yang S, et al. Phage display-derived oligopeptide-functionalized probes for in vivo specific photoacoustic imaging of osteosarcoma. Nanomedicine. United States; 2017;13:111-21.
16. Sato AK, Viswanathan M, Kent RB, Wood CR. Therapeutic peptides: technological advances driving peptides into development. Curr Opin Biotechnol. England; 2006;17:638-42.
17. Kang CK, Jayasinha V, Martin PT. Identification of peptides that specifically bind Abeta1-40 amyloid in vitro and amyloid plaques in Alzheimer's disease brain using phage display. Neurobiol Dis. United States; 2003;14:146-56.
18. Orner BP, Liu L, Murphy RM, Kiessling LL. Phage display affords peptides that modulate beta-amyloid aggregation. J Am Chem Soc. 2006;128:11882-9.
19. Zi Y, Zhu J, Hu L, Wang M, Huang W. Nanoengineering of Tin Monosulfide (SnS)-Based Structures for Emerging Applications. Small Sci. 2022;2:2100098.
20. Huang W, Zhu J, Wang M, Hu L, Tang Y, Shu Y, et al. Emerging Mono-Elemental Bismuth Nanostructures: Controlled Synthesis and Their Versatile Applications. Adv Funct Mater. 2021;31:2007584.
21. Cheng Y, Muroski ME, Petit DCMC, Mansell R, Vemulkar T, Morshed RA, et al. Rotating magnetic field induced oscillation of magnetic particles for in vivo mechanical destruction of malignant glioma. J Control Release. Netherlands; 2016;223:75-84.
22. Sun C, Lee JSH, Zhang M. Magnetic nanoparticles in MR imaging and drug delivery. Adv Drug Deliv Rev. Netherlands; 2008;60:1252-65.
23. Tran N, Webster TJ. Magnetic nanoparticles: biomedical applications and challenges. J Mater Chem [Internet]. The Royal Society of Chemistry; 2010;20:8760-7. Available from: http://dx.doi.org/10.1039/COJM00994F
24. Farzin A, Etesami SA, Quint J, Memic A, Tamayol A. Magnetic Nanoparticles in Cancer Therapy and Diagnosis. Adv Healthc Mater. 2020;9:1901058.
25. Chowdhury UR, Madden BJ, Charlesworth MC, Fautsch MP. Proteome analysis of human aqueous humor. Invest Ophthalmol Vis Sci. United States; 2010;51:4921-31.
26. Heys JJ, Barocas VH. A boussinesq model of natural convection in the human eye and the formation of Krukenberg's spindle. Ann Biomed Eng. United States; 2002;30:392-401.
27. Hauser AK, Wydra RJ, Stocke NA, Anderson KW, Hilt JZ. Magnetic nanoparticles and nanocomposites for remote controlled therapies. J Control Release [Internet]. 2015;219:76-94. Available from: http://www.sciencedirect.com/science/article/pii/S0168365915301395
28. Singh N, Jenkins GJS, Asadi R, Doak SH. Potential toxicity of superparamagnetic iron oxide nanoparticles (SPION). Nano Rev [Internet]. 2010 Sep. 21. CoAction Publishing; 2010;1:10.3402/nano.vli0.5358. Available from: https://pubmed.ncbi.nlm.nih.gov/22110864
29. Zarghami N, Khrapitchev AA, Perez-Balderas F, Soto MS, Larkin JR, Bau L, et al. Optimization of molecularly targeted MRI in the brain: empirical comparison of sequences and particles. Int J Nanomedicine. New Zealand; 2018; 13:4345-59.
30. Chirita M, Kiss ML, Savii C. Magnetite, Properties and Modern Biomedical Applications. In: Ward J, editor. Magn Synth Uses Biol Occur. Nova Science Publishers, Inc., 2014. p. 119-81.
31. Reddy LH, Arias JL, Nicolas J, Couvreur P. Magnetic nanoparticles: design and characterization, toxicity and biocompatibility, pharmaceutical and biomedical applications. Chem Rev. United States; 2012;112:5818-78.
32. Daldrup-Link HE. Ten things you might not know about iron oxide nanoparticles. Radiology. United States; 2017;284:616-29.
33. Stamer WD, Clark AF. The many faces of the trabecular meshwork cell. Exp Eye Res. England; 2017;158:112-23.
34. Polansky JR, Wood IS, Maglio MT, Alvarado JA. Trabecular meshwork cell culture in glaucoma research: evaluation of biological activity and structural properties of human trabecular cells in vitro. Ophthalmology. United States; 1984;91:580-95.
35. Raju HB, Hu Y, Vedula A, Dubovy SR, Goldberg JL. Evaluation of magnetic micro-and nanoparticle toxicity to ocular tissues. PLOS One. 2011;6.
36. Damji KF, Konstas AGP, Liebmann JM, Hodge WG, Ziakas NG, Giannikakis S, et al. Intraocular pressure following phacoemulsification in patients with and without exfoliation syndrome: a 2 year prospective study. Br J Ophthalmol [Internet]. 2006 May 3. BMJ Group; 2006;90:1014-8. Available from: https://pubmed.ncbi.nlm.nih.gov/16672324
37. V Tran T, Mansouri K, Mermoud A. Goniowash: a new surgical approach combined with cataract surgery to lower intraocular pressure in pseudoexfoliation syndrome. Int Ophthalmol. 2021;41:1563-71.
38. Soetaert F, Korangath P, Serantes D, Fiering S, Ivkov R. Cancer therapy with iron oxide nanoparticles: Agents of thermal and immune therapies. Adv Drug Deliv Rev. 2020; 163-164:65-83.
39. Materón EM, Miyazaki CM, Carr O, Joshi N, Picciani PHS, Dalmaschio CJ, et al. Magnetic nanoparticles in biomedical applications: A review. Appl Surf Sci Adv. 2021;6:100163.
40. Kelly KA, Waterman P, Weissleder R. In vivo imaging of molecularly targeted phage. Neoplasia. United States; 2006;8:1011-8.
41. Presolski SI, Hong VP, Finn MG. Copper-Catalyzed Azide-Alkyne Click Chemistry for Bioconjugation. Curr Protoc Chem Biol. United States; 2011;3:153-62.
42. Saltzman WM. Tissue engineering: engineering principles for the design of replacement organs and tissues. Oxford University Press; 2004.

1. A peptide having specific affinity to exfoliation syndrome (XFS) deposits in a mammalian eye comprising or consisting essentially of an amino acid sequence comprising LPSYNLHPHVPP [SEQ ID NO. 1] or; IPLLNPGSMQLS [SEQ ID NO. 2], or variants or modified derivatives thereof, or a pharmaceutically acceptable salt of the peptide.

2. A magnetic particle-peptide conjugate comprising a peptide of claim 1 and a magnetic particle.

3. The conjugate of claim 2 wherein the magnetic particle comprises iron oxide.

4. The conjugate of claim 3, wherein the magnetic particle comprises magnetite, maghemite or ferrite.

5. The conjugate of claim 2, wherein the magnetic particles is substantially spherical and/or is about 1 micron in diameter.

6. A method for the treatment of XFS or XFG in a mammalian eye comprising targeting of XFS deposits with a magnetic particle (MP)-peptide conjugate of claim 2, and displacing the XFS deposits by application of a magnetic field.

7. The method of claim 6 which is applied to a tissue within an anterior segment of the eye.

8. The method of claim 6, comprising the further step of irrigating or flushing the eye following magnetic displacement.

9. The method of claim 6, wherein the magnetic field strength is between about 1000 G to about 10,000 G.

10. The method of claim 9 wherein the magnetic field is a low frequency rotating field.

11. Use of a magnetic particle-conjugate of claims 2, to target and remove XFS deposits in a mammalian eye.

12. The method of claim 6 wherein the mammalian eye is a human eye.

13. The use of claim 11 wherein the mammalian eye is a human eye.
